(19) United States
(12) Patent Application Publication
(10) Pub. No.: US 2016/0271121 A1
(43) Pub. Date: Sep. 22, 2016

(54) ANTIFUNGAL COMBINATION THERAPY

(71) Applicant: Gavis Pharmaceuticals, Somerset, NJ (US)

(72) Inventors: Bala Chandran Nayar, Somerset, NJ (US); Aman Trehan, Somerset, NJ (US)

(73) Assignee: GAVIS PHARMACEUTICALS, Somerset, NJ (US)

(21) Appl. No.: 14/658,521

(22) Filed: Mar. 16, 2015

(51) Int. Cl. A61K 31/445; A61K 45/06; A61K 9/00; A61K 31/4418

(52) U.S. Cl. CPC A61K 31/445; A61K 31/4418; A61K 45/06; A61K 9/0014

(57) ABSTRACT

A novel combination of two antifungal agents is provided. The combination contains ciclopirox and a triazole containing antifungal agent or salts thereof. The invention also provides a method of treating onychomycosis using said antifungal agent combination.

### BACKGROUND OF THE INVENTION

(a) Field of the Invention

The present invention is directed to antifungal combination therapy comprising the use of two antifungal agents, ciclopirox and a triazole antifungal agent or salts thereof. More particularly, the invention relates to an antifungal combination therapy comprising the use of ciclopirox and a triazole antifungal agent, particularly efinaconazole or salts thereof for treating fungal infections such as onychomycosis.

(b) Description of the Related Art

Onychomycosis is a disease of the nail caused by yeast, dermatophytes, or other molds, and represents approximately 50% of all nail disorders. Toenail infection accounts for approximately 80% of onychomycosis incidence, while fingernails are affected in about 20% of the cases. Dermatophytes are the most frequent cause of nail plate invasion, particularly in toenail onychomycosis. Onychomycosis caused by a dermatophyte is termed Tinea unguium. Trichophyton rubrum is by far the most frequently isolated dermatophyte, followed by T. mentagrophytes. Distal subungual onychomycosis is the most common presentation of tinea unguium, with the main site of entry through the hyponychium (the thickened epidermis underneath the free distal end of a nail) progressing in time to involve the nail bed and the nail plate. Discoloration, onycholysis, and accumulation of subungual debris and nail plate dystrophy characterize the disease. The disease adversely affects the quality of life of its victims, with subject complaints ranging from unsightly nails and discomfort with footwear, to more serious complications including secondary bacterial infections.

In most cases, onychomycosis is associated with limited treatment options that are effective in achieving complete clearance. In addition, recurrence rates are high in the subset of treated patients who have been effectively cleared, usually with an oral antifungal agent.

Many methods are known for the treatment of fungal infections, including the oral and topical use of antibiotics (e.g., nystatin and amphotericin B), imidazole anti-fungal agents such as miconazole, clotrimazole, fluconazole, econazole and sulconazole, and non-imidazole fungal agents such as the allylamine derivatives terbinafine and naftifine, and the benzylamine butenafine. The existing topical antifungals are not associated with dangerous adverse events, as they rarely penetrate the systemic circulation and gain a significant concentration in the body.

However, onychomycosis has proven to be resistant to most treatments. Nail fungal infections reside in an area difficult to access by conventional topical treatment and anti-fungal drugs cannot readily penetrate the nail plate to reach the infection sites under the nail. Therefore, onychomycosis has traditionally been treated by oral administration of anti-fungal drugs; however, clearly this is undesirable due to the potential for side effects of such drugs, in particular those caused by the more potent anti-fungal drugs such as itraconazole and ketoconazole. An alternative method of treatment of onychomycosis is by removal of the nail before treating with a topically active anti-fungal agent; such a method of treatment is equally undesirable. Systemic antimycotic agents require prolonged use and have the potential for significant side effects. Topical agents have usually been of little benefit, primarily because of poor penetration of the anti-fungal agents into and through the nail mass.

U.S. Pat. No. 5,030,619 discloses an antifungal or antimycotic composition comprising therapeutically effective amounts of nikkomycin and echinocandin B.

U.S. Pat. No. 6,875,740 discloses antifungal combination use of azole and polyene antifungals in combination with a lipopeptide compound antifungal agent.

U.S. Pat. No. 8,552,042 discloses a method of treating candidiasis by using a combination of an antimycotic agent and an epithelial cell or endothelial cell adhesion inhibitor such as iloprost, cicaprost, ticlopidine or clopidogrel.

International Application WO 88/06884 teaches treating nail mycosis with a pharmaceutically effective amount of a topical antimycotic such as an imidazole compound, optionally with an antiseptic such as salicylic acid.

U.S. Pat. No. 6,416,749 discloses a method of treating onychomycosis using salicylic acid and optionally in combination with retinoid compounds such as tretinoin, adapalene, manoalide, retinol, and tretinate.

There has been a noticeable absence of medical therapies available for onychomycosis. Since the introduction of topical ciclopirox (8% nail lacquer), no new effective agent has been introduced for more than 10 years. Fortunately, newer agents and formulations have been under formal development. While patients might prefer a topical therapy, efficacy with ciclopirox 8% nail lacquer is insufficient. The poor therapeutic outcomes achieved with ciclopirox 8% nail lacquer were not unexpected as the cure rates achieved in the clinical trials were unimpressive, despite concomitant nail debridement, which was an integral part of the pivotal trials with ciclopirox 8% nail lacquer.

Ciclopirox is a known topical antifungal or antimycotic agent belonging to the chemical class of hydroxypyridones and not related to azoles or any other class of antifungal agents. It has the chemical name 6-cyclohexyl-1-hydroxy-4-methyl-2(1H)-pyridone and structure as follows:

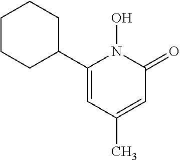

Ciclopirox 8% solution has been approved for the treatment of onychomycosis in the United States and Canada. While ciclopirox shows a degree of efficacy against onychomycosis for certain patient populations, it has proven not to be universally effective against the illness.

U.S. Pat. No. 6,495,124 discloses a composition for the treatment of fungal infections of nails, comprising at least one antifungal agent such as ciclopirox or econazole, and a plasticizing and penetration enhancing compound.

U.S. Pat. No. 6,224,887 discloses a composition for the treatment or prevention of fungal infections of nails comprising at least one antifungal agent effective for prevention of onychomycosis selected from polyenes, allylamines, imidazoles, triazoles, ciclopirox, undecylenic acid, and amorolfine, a penetration enhancing agent, a water insoluble film forming polymer and volatile solvent.

US Application Publication No. 20100273834 discloses a composition for treatment of onychomycosis comprising ciclopirox and urea.

US Application Publication No. 20100261695 discloses a composition for treatment of onychomycosis comprising ciclopirox and at least one other antifungal agent. The publication discloses several conventional antifungal agents to be used in combination with ciclopirox for onychomycosis treatment.

Canadian Patent Application No. CA 2704425 A1 discloses a combination of fungicidal triazole antifungal agents together with a pyrion compound including ciclopirox.

Recently, a new antifungal agent, efinaconazole, belonging to the triazole class of antifungal agents has been developed. Efinaconazole is specifically developed for the topical treatment of distal and lateral subungual onychomycosis (DLSO). It has the chemical name (2R,3R)-2-(2,4-difluorophenyl)-3-(4-methylenepiperidin-1-yl)-1-(1H-1,2,4-triazol-1-yl)butan-2-ol and structure as follows:

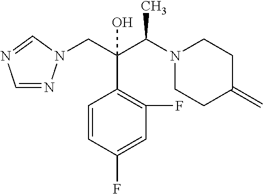 [SMILES: C=C1CCN([C@H](C)[C@](O)(Cn2cncn2)c2ccc(F)cc2F)CC1]

Efinaconazole is an inhibitor of sterol 14α-demethylase. In broth dilution tests in vitro against reference strains, it was more potent than terbinafine, ciclopirox, itraconazole, and amorolfine.

In Phase 3 clinical trials, a 10% nail solution of efinaconazole was applied once daily for 48 weeks followed by a four-weeks of washout period. The trial outcome measures were evaluated at week 52 and results from these evaluations demonstrated that the formulation was superior to the vehicle for all outcome measures. A comparison of the mycological cure rates for efinaconazole was made to other antifungal agents, itraconazole, terbinafine and ciclopirox. The mycological cure rate for 48 weeks of topical efinaconazole was more than that for 48 weeks of topical ciclopirox and comparable to 12 weeks of oral itraconazole. (Skin Therapy Letter. 2014; 19(1); Efinaconazole: A new topical treatment for onychomycosis.)

U.S. Pat. No. 7,214,506 discloses triazole-containing compounds including efinaconazole for treating onychomycosis.

Despite the currently available mono and combination therapies, a new, alternate and effective drug combination for management of onychomycosis is still required. A need of such new combination treatment options for onychomycosis also exists in the art which can effectively penetrate as well as treat the nail infection in shortest possible duration. These and other needs are addressed by the current invention.

### SUMMARY OF THE INVENTION

The present invention generally relates to the treatment of onychomycosis. More specifically, it relates to a combination therapy of ciclopirox and a triazole containing antifungal agent or salts thereof for the treatment of onychomycosis and related conditions.

In a first aspect, the invention provides a combination of ciclopirox and efinaconazole, or salts thereof.

In a related aspect, the invention provides a combination of ciclopirox and efinaconazole, or salts thereof as the sole active ingredients.

In a related aspect, the invention consists of or consists essentially of a combination of ciclopirox and efinaconazole, or salts thereof as the sole active ingredients.

In another aspect, the invention provides a pharmaceutical composition comprising (a) ciclopirox or salts thereof, (b) efinaconazole or salts thereof; and (c) a pharmaceutically acceptable excipient. The composition may be in the form suitable for topical administration, such as solution, gel, cream, lotion, tincture, or ointment. The ciclopirox and efinaconazole may be the sole active ingredients.

In another aspect, the invention provides a topical pharmaceutical solution comprising (a) ciclopirox or salts thereof, (b) efinaconazole or salts thereof; and (c) a pharmaceutically acceptable excipient.

In another aspect, the invention provides a topical pharmaceutical solution for the treatment of a disorder of the nail or nail bed comprising (a) ciclopirox or salts thereof, (b) efinaconazole or salts thereof, (c) a solvent that is volatile and/or that rapidly penetrates a nail following application onto the surface of the nail, (d) a wetting agent, and (e) optionally a non-volatile solvent that is dissolved, suspended, dispersed, or emulsified within the solvent.

In another aspect, the invention provides a combination of ciclopirox, efinaconazole or salts thereof and one or more additional antifungal agents.

In another aspect, the invention provides a pharmaceutical composition comprising (a) efinaconazole, (b) ciclopirox, and (c) a pharmaceutically acceptable excipient, which composition exhibits excellent storage stability.

In another aspect, the invention provides a method of treating onychomycosis in a human, comprising administering to the animal a therapeutically effective amount of ciclopirox and efinaconazole or salts thereof.

In another aspect, the invention provides a method of delivering efinaconazole and ciclopirox from the dorsal layer of the nail plate to the nail bed. The method comprises contacting said cell with the combination of ciclopirox and efinaconazole or salts thereof capable of penetrating the nail plate, under conditions sufficient to penetrate said nail plate, and thereby delivering said combination.

In another aspect, the invention provides a method for treating onychomycosis in a patient suffering from the disease. The method involves topically administering a composition to at least one toe or fingernail of the patient, wherein the composition includes ciclopirox and efinaconazole or salts thereof.

Still other aspects and advantages of the invention will be apparent from the following detailed description of the invention.

### DETAILED DESCRIPTION OF THE INVENTION

The invention addresses the need for a novel and effective combination therapy for onychomycosis. The present invention relates to combination therapies for the treatment of onychomycosis and related compositions. The compositions include ciclopirox and a triazole containing antifungal agent or salts thereof. Preferably, the triazole containing antifungal agent is efinaconazole.

The inventors have observed that the combination of ciclopirox and a triazole containing antifungal agent, and particularly efinaconazole, acts synergistically, resulting in better penetration of both antifungal gents in the nail bed and fungal infection, and improving symptoms of the infection relatively faster than that achieved when the two antifungal agents are applied individually. The inventors have also observed that antifungal effect of said combination is more than the combined antifungal effect of the individual antifungal agents.

The term “topical administration” refers to the application of a pharmaceutical agent to the external surface of the skin, nail, hair, claw or hoof, such that the agent crosses the external surface of the skin, nail, hair, claw or hoof and enters the underlying tissues. Topical administration includes application of the composition to intact skin, nail, hair, claw or hoof, or to a broken, raw or open wound of skin, nail, hair, claw or hoof. Topical administration of a pharmaceutical agent can result in a limited distribution of the agent to the skin and surrounding tissues or, when the agent is removed from the treatment area by the bloodstream, can result in systemic distribution of the agent.

“MIC”, or “minimum inhibitory concentration”, is the point where the compound or combination of compounds stops more than 90% of cell growth relative to an untreated control.

The term “pharmaceutically acceptable excipients” refers to solvents, preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient.

The term “volatile” when referring to the solvent means that the solvent is a compound that evaporates from the surface of the nail when applied. Volatile solvents are compounds which have a measurable vapor pressure, and preferably are compounds that have a vapor pressure of greater than about 100 Pa at room temperature. Examples of volatile solvents include: acetone, 2-amino-2-methyl-1-propanol, 1,2-butanediol, 1,4-butanediol, 2-butanol, cyclomethicone-4, cyclomethicone-5, cyclomethicone-6, ethanol, ethyl acetate, n-heptane, isobutanol, isopropyl alcohol, 1-propanol, 2-propanol, and water.

As used herein, the term “penetrating” when referring to the solvent means that the solvent is a compound that rapidly penetrates into a nail when applied to the surface of the nail so that, after 10 minutes following the application of a thin layer of the solvent onto the surface of a nail, no more than 10% of the applied amount remains on the nail surface. The term “penetrating” thus includes both volatile and non-volatile solvents.

The abbreviations used herein generally have their conventional meaning within the chemical and biological arts.

In the present invention, the antifungal agents are, preferably, present in the free form, e.g., as acid or base, rather than in the form of their salts. For example, ciclopirox may be used in the form of a free base or its olamine salt. “Ciclopirox” refers to 6-cyclohexyl-1-hydroxy-4 methyl-2 (1H)-pyridone and “Ciclopirox olamine” refers to the 2-aminoethanol salt of Ciclopirox.

Embodiments of the invention are directed to a method of inhibiting the growth of a microorganism, or killing a microorganism, or both, comprising contacting the microorganism with ciclopirox and efinaconazole. Microorganisms are members selected from fungi, yeast, viruses, bacteria and parasites. In another exemplary embodiment, the microorganism is inside, or on the surface of an animal. In an embodiment, the animal is a human.

Embodiments of the invention are also directed to a method of treating or preventing an infection, or both. The method includes administering to the animal a therapeutically effective amount of ciclopirox and efinaconazole or salts thereof, sufficient to treat or prevent said infection. In another exemplary embodiment, the infection is a member selected from a systemic infection, a cutaneous infection, and an ungual or periungual infection.

Embodiments are also directed to a method of treating or preventing an ungual and/or periungual infection. The method includes administering to the animal a therapeutically effective amount of ciclopirox and efinaconazole or salts thereof, sufficient to treat or prevent said infection.

Embodiments are also directed to a method of treating or preventing onychomycosis. The method includes administering to the animal a therapeutically effective amount of a pharmaceutical formulation of the invention, sufficient to treat or prevent onychomycosis. In another exemplary embodiment, the method includes administering the pharmaceutical formulation of the invention at a site which is a member selected from the skin, nail, hair, hoof, claw and the skin surrounding the nail, hair, hoof and claw.

Embodiments are also directed to a method of delivering ciclopirox and efinaconazole or salts thereof from the dorsal layer of the nail plate to the nail bed.

This method comprises contacting the cell with ciclopirox and efinaconazole or salts thereof capable of penetrating the nail plate, under conditions sufficient to penetrate the nail.

In an embodiment, the invention is a pharmaceutical composition which includes: (a) ciclopirox or salts thereof, (b) efinaconazole or salts thereof; and (c) a pharmaceutically acceptable excipient.

The composition is typically a solution, cream, ointment, foam or spray, containing any concentration of ciclopirox and efinaconazole that produces a desirable pharmaceutical effect is suitable. Non-limiting examples of other Ciclopirox or efinaconazole concentrations include a 3% to 15% solution or cream. In an embodiment, ciclopirox and efinaconazole concentrations include 5%, 6%, 7%, 9%, 10%, 11%, 12%, and 13%.

Where a single additional antifungal agent is included in the composition, it is typically included at a concentration of 0.5%, 1%, 1.5%, 2%, 2.5%, 3%, 3.5%, 4%, 4.5% or 5% in the solution or cream.

Where two additional antifungal agents are included in the composition, their combined concentration is typically 0.5%, 1%, 1.5%, 2%, 2.5%, 3%, 3.5%, 4%, 4.5% or 5% in the solution or cream.

Additional antifungal agents which can be included in the combination of the present invention are typically selected from one or more of the following list: terbinafne; itraconazole; ketoconazole; fluconazole; derivatives of fuconazole; oxiconazole; sulconazole; clotrimazole; miconazole; econazole; azanidazole; bifonazole; butoconazole; chlormidazole; fenticonazole; imazalil; isoconazole; neticonazole; sertaconazole; tioconazole; naftifne; griseofulvin; amorolfine; sodium pyrithione, bifonazole/urea; and, propylene glycol-urea-lactic acid.

The pharmaceutical compositions of the invention can take a variety of forms adapted to the chosen route of administration.

Those skilled in the art will recognize various synthetic methodologies that may be employed to prepare non-toxic pharmaceutical compositions incorporating the combination described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The compositions of the invention are administered topically in dosage unit formulations containing conventional non-toxic pharmaceutically acceptable carriers, adjuvants and solvents. It is further understood that the best method of administration may be a combination of methods.

Embodiments are also directed to topical pharmaceutical compositions which include: (a) ciclopirox or salts thereof, (b) efinaconazole or salts thereof; and (c) a pharmaceutically acceptable excipient.

The compositions of the present invention comprise fluid or semi-solid excipients that may include but are not limited to polymers, thickeners, buffers, neutralizers, chelating agents, preservatives, surfactants or emulsifiers, antioxidants, waxes or oils, emollients, sunscreens, and a solvent or mixed solvent system. The solvent or mixed solvent system is important to the formation because it is primarily responsible for dissolving the drug. The best solvent or mixed solvent systems are also capable of maintaining clinically relevant levels of the drug in solution despite the addition of a poor solvent to the formulation. The topical compositions useful in the subject invention can be made into a wide variety of product types. These include, but are not limited to, lotions, creams, gels, sticks, sprays, ointments, pastes, foams, mousses, and cleansers. These product types can comprise several types of carrier systems including, but not limited to particles, nanoparticles, and liposomes. If desired, disintegrating agents can be added, such as the cross-linked polyvinyl pyrrolidone, agar or alginic acid or a salt thereof such as sodium alginate. Techniques for formulation and administration can be found in Remington: The Science and Practice of Pharmacy, supra. The formulation can be selected to maximize delivery to a desired target site in the body.

Lotions, which are preparations that are to be applied to the skin, nail, hair, claw or hoof surface without friction, are typically liquid or semi-liquid preparations in which finely divided solid, waxy, or liquid are dispersed. Lotions will typically contain suspending agents to produce better dispersions as well as compounds useful for localizing and holding the active agent in contact with the skin, nail, hair, claw or hoof, e.g., methylcellulose, sodium carboxymethyl-cellulose, or the like.

Creams containing the active agent for delivery according to the present invention are viscous liquid or semisolid emulsions, either oil-in-water or water-in-oil. Cream bases are water-washable, and contain an oil phase, an emulsifier and an aqueous phase. The oil phase is generally comprised of petrolatum or a fatty alcohol, such as cetyl- or stearyl alcohol; the aqueous phase usually, although not necessarily, exceeds the oil phase in volume, and generally contains a humectant. The emulsifier in a cream formulation, as explained in Remington: The Science and Practice of Pharmacy, supra, is generally a nonionic, anionic, cationic or amphoteric surfactant.

Gel formulations can also be used in connection with the present invention. As will be appreciated by those working in the field of topical drug formulation, gels are semisolid. Single-phase gels contain organic macromolecules distributed substantially uniformly throughout the carrier liquid, which is typically aqueous, but also may be a solvent or solvent blend.

Ointments, which are semisolid preparations, are typically based on petrolatum or other petroleum derivatives. As will be appreciated by the ordinarily skilled artisan, the specific ointment base to be used is one that provides for optimum delivery for the active agent chosen for a given formulation, and, preferably, provides for other desired characteristics as well, e.g., emolliency or the like. As with other carriers or solvents, an ointment base should be inert, stable, non-irritating and non-sensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases, emulsifiable bases, emulsion bases, and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy, supra, for further information.

Liquid forms, such as lotions suitable for topical administration or suitable for cosmetic application, may include a suitable aqueous or nonaqueous solvents with buffers, suspending and dispensing agents, thickeners, penetration enhancers, and the like. Solid forms such as creams or pastes or the like may include, for example, any of the following ingredients: water, oil, alcohol or grease as a substrate with surfactant, polymers such as polyethylene glycol, thickeners, solids and the like. Liquid or solid formulations may include enhanced delivery technologies such as liposomes, microsomes, microsponges and the like.

In an embodiment, the pharmaceutical composition is in the form of a simple solution. In an embodiment, the simple solution includes an alcohol. In a further embodiment, the simple solution includes alcohol and water. In a further embodiment, the alcohol is ethanol, ethylene glycol, propanol, polypropylene glycol, isopropanol or butanol. In another exemplary embodiment, the simple solution is a member selected from about 10% polypropylene glycol and about 90% ethanol; about 20% polypropylene glycol and about 80% ethanol; about 30% polypropylene glycol and about 70% ethanol; about 40% polypropylene glycol and about 60% ethanol; about 50% polypropylene glycol and about 50% ethanol; about 60% polypropylene glycol and about 40% ethanol; about 70% polypropylene glycol and about 30% ethanol; about 80% polypropylene glycol and about 20% ethanol; or about 90% polypropylene glycol and about 10% ethanol.

Additives for topical formulations are well-known in the art, and may be added to the topical composition, as long as they are pharmaceutically acceptable and not deleterious to the epithelial cells or their function. Further, they should not cause deterioration in the stability of the composition. For example, additives that may be used include one or more of inert fillers, anti-irritants, tackifiers, excipients, fragrances, opacifiers, antioxidants, wetting agents, gelling agents, stabilizers, surfactants, emollients, colouring agents, preservatives, buffering agents, other permeation enhancers, and other conventional components of topical or transdermal delivery formulations as are known in the art.

The topical pharmaceutical compositions may also comprise suitable nail penetration enhancers. Examples of nail penetration enhancers include mercaptan compounds, acetyl cysteine, sulfites and bisulfites, keratolytic agents and surfactants. Nail penetration enhancers suitable for use in the invention are described in greater detail in Malhotra et al., J. Pharm. Sci., 91:2, 312-323 (2002), which is incorporated herein by reference in its entirety and in particular for its disclosure of penetration enhancers suitable for nail penetration.

In an embodiment, the topical pharmaceutical solution for the treatment of a disorder of the nail or nail bed comprises: (a) ciclopirox or salts thereof, (b) efinaconazole or salts thereof, (c) a solvent that is volatile and/or that rapidly penetrates a nail following application onto the surface of the nail, (d) a wetting agent, and (e) optionally a non-volatile solvent that is dissolved, suspended, dispersed, or emulsified within the solvent.

The solvent is one in which the constituents of the composition of the invention can be dissolved, suspended, disbursed, or emulsified. The constituents of the composition may be all within a single phase in the solvent. For example, the active ingredients, wetting agent, and the non-volatile phase may be dissolved in single solvent. Alternatively, the constituents may occupy separate phases within the solvent. For example, the active ingredients may be dissolved in one solvent and the other constituents may be suspended, dispersed, or emulsified in another solvent. For another example, the active ingredients may be dissolved in one solvent which is suspended, dispersed, or emulsified in another solvent, with the remaining constituents being dissolved in either the first solvent or the second solvent. Preferably, but not necessarily, the active ingredients, wetting agent, and non-volatile phase are all miscible in the solvent.

Examples of suitable solvents include one or more of water, alcohols, polyols, ethers, esters, aldehydes, ketones, fatty acids, fatty alcohols, and fatty esters. Specific examples of suitable solvents include ethanol; 3-propanediol; 1,2-butanediol; 1,2,3-propanetriol; 1,3-butanediol; 1,4-butanediol; isopropyl alcohol; and 2-amino-2-methyl-1-propanol. In a preferred embodiment, the solvent is an alcohol, and most preferably a linear or branched aliphatic lower alcohol, such as methanol, ethanol, propanol, or isopropanol.

The wetting agent is a chemical compound that reduces the surface tension of liquid compositions and does not build viscosity. Any surfactant or group of surfactants that is suitable for dermatologic applications is suitable for the invention. Such surfactants may function as wetting agents in the compositions of the invention, and as emulsifiers or solubilizers. The surfactants may be nonionic, anionic, cationic, zwitterionic, amphoteric, or ampholytic surfactants.

The non-volatile solvent is a solvent that may or may not be soluble or miscible in the solvent of the composition. Drugs of the composition are preferably, but not necessarily, soluble in the non-volatile solvent.

Suitable non-volatile solvents may be an ester of the formula RCO—OR′, wherein R and R′ may be identical or different and each of R and R′ represents a linear or branched chain of an alkyl, alkenyl, alkoxycarbonylalkyl, or alkoxycarbonyloxyalkyl radical having from 1 to 25 carbon atoms, preferably from 4 to 20 carbon atoms. The non-volatile solvent may be a glyceryl ester of a fatty acid, such as fatty esters of natural fatty acids or triglycerides of animal or plant origin. The non-volatile solvent may be a fatty acid glyceride, including synthetic or semi-synthetic glyceryl esters, such as fatty acid mono-, di-, or triglycerides, which are oils or fats. The non-volatile solvent may be a non-volatile hydrocarbon, such as paraffins, isoparaffins, and mineral oil. The non-volatile solvent may be a guerbet ester. The non-volatile solvent may be a non-volatile silicone, provided that the presence of the non-volatile silicone in the composition does not result in the formation of a hard polymeric film upon application of the composition onto a nail. Included within such non-film forming silicones are polyorganosiloxane compounds that have the formula [R₁SiOR₂]ₙ wherein n>6 and R₁ and R₂ are alkyl groups that may be the same or different, and which compound may or may not have a measurable vapour pressure under ambient conditions.

Other examples of suitable non-volatile solvents include squalane, dibutyl sebacate, isopropyl laurate, isopropyl myristate, isopropyl palmitate, isopropyl stearate, myristyl alcohol, oleyl alcohol, oleic acid, lauryl lactate, myristyl lactate, mixed C12-15 alkyl lactates, diisopropyl adipate, octyldodecanol, caproic acid, caprylic acid, capric acid, lauryl benzoate, myristyl benzoate, mixed C12-15 alkyl benzoates, benzyl benzoate, tridecyl neopentanoate, light mineral oil, mineral oil, and alpha terpineol. Examples of suitable non-volatile solvents for hydrophilic drugs include diethylene glycol monoethyl ether, n-methylpyrrolidone, dimethyl sulfoxide, ethyl lactate, hexylene glycol, glycerol, benzyl alcohol and glycerol triacetate.

In an embodiment, the topical solution of ciclopirox and efinaconazole in accordance of the present invention comprises citric acid, alcohol, butylated hydroxytoluene, C₁₂₋₁₅ alkyl lactate, cyclomethicone, diisopropyl adipate, and disodium editate.

### Example 1

### Ciclopirox and Efinaconazole Topical Solution

TABLE 1

| Sr. | | Formula 1 | Formula 2 | Formula 3 | Formula 4 |
|---|---|---|---|---|---|
| No. | Ingredients | (% w/w) | (% w/w) | (% w/w) | (% w/w) |
| 1 | Ciclopirox (base) | 4 | 6 | 8 | 10 |
| 2 | Efinaconazole | 2 | 4 | 5 | 8 |
| 3 | Alcohol | 10 | 15 | 20 | 30 |
| 4 | Isopropyl Alcohol | 40 | 0 | 0 | 20 |
| 5 | Propylene Glycol | 17 | 33 | 10 | 10 |
| 6 | Ethyl Acetate | 5 | 10 | 15 | 20 |
| 7 | Amyl Acetate | 2 | 2 | 2 | 2 |
| 8 | Dimethyl | 20 | 30 | 40 | 0 |
| | Sulfoxide | | | | |
| | Total | 100 | 100 | 100 | 100 |

(Thickening agents and fragrances are optionally added).

Process:

Alcohol, Isopropyl Alcohol, Propylene Glycol, Ethyl Acetate, Amyl acetate and Dimethyl Sulfoxide were mixed in a suitable stainless steel container fixed with Lighten′ mixer until a clear solution was obtained. Ciclopirox was added to the solvent mixture until dissolved. Efinaconazole was added to the solvent mixture and mixed well until dissolved and a clear solution was obtained.

In an alternate process, Ciclopirox and Efinaconazole were dissolved in alcohol and isopropyl alcohol first, then the other ingredients were added and dissolved until a clear solution was obtained.

1. A topical pharmaceutical composition comprising:
(a) ciclopirox or a salt thereof, (b) efinaconazole or a salt thereof; and (b) one or more pharmaceutically acceptable excipients.

2. The composition of claim 1, wherein the salt of ciclopirox is ciclopirox olamine.

3. The composition of claim 1, wherein the composition further comprises one or more additional antifungal agents.

4. The composition of claim 3, wherein the antifungal agents are selected from the group consisting of: terbinafine, itraconazole, ketoconazole, fluconazole, derivatives of fluconazole, oxiconazole, sulconazole, clotrimazole, miconazole, econazole, azanidazole, bifonazole, butoconazole, chlormidazole, fenticonazole, imazalil, isoconazole, neticonazole, sertaconazole, tioconazole, naftifne, griseofulvin, amorolfine, sodium pyrithione, bifonazole/urea; and propylene glycol-urea-lactic acid.

5. The composition of claim 1, wherein the composition is in the form of a solution, gel, cream, lotion, tincture, or ointment.

6. The composition of claim 1, wherein the composition is in the form of a solution for topical application.

7. The composition of claim 1, wherein the composition consists essentially of a) ciclopirox or a salt thereof and efinaconazole or a salt thereof to treat onychomycosis; and (b) one or more pharmaceutically acceptable excipients.

8. The composition of claim 7, wherein the composition consists of ciclopirox or a salt thereof and efinaconazole or a salt thereof as the sole active agents to treat onychomycosis.

9. The composition of claim 1, wherein the composition consists of a) ciclopirox or a salt thereof, (b) efinaconazole or a salt thereof; and (b) one or more pharmaceutically acceptable excipients.

10. The composition of claim 9, wherein the composition consists of ciclopirox or a salt thereof and efinaconazole or a salt thereof as the active agents to treat onychomycosis.

11. The composition of claim 9, wherein the composition consists of ciclopirox or a salt, efinaconazole or a salt, ethanol, isopropyl alcohol, propylene glycol, ethyl acetate, amyl acetate and dimethyl sulfoxide.

12. The composition of claim 11, wherein the composition consists of
4-10% w/w ciclopirox or a salt;
2-8% w/w efinaconazole or a salt;
10-30% w/w ethanol;
0-40% w/w isopropyl alcohol;
10-33% w/w propylene glycol;
5-20% w/w ethyl acetate;
2% w/w amyl acetate; and
0-40% w/w dimethyl sulfoxide.

13. The composition of claim 11, wherein the composition consists of
4% w/w ciclopirox or a salt;
2% w/w efinaconazole or a salt;
10% w/w ethanol;
40% w/w isopropyl alcohol;
17% w/w propylene glycol;
5% w/w ethyl acetate;
2% w/w amyl acetate; and
20% w/w dimethyl sulfoxide.

14. The composition of claim 11, wherein the composition consists of
10% w/w ciclopirox or a salt;
8% w/w efinaconazole or a salt;
30% w/w ethanol;
20% w/w isopropyl alcohol;
10% w/w propylene glycol;
20% w/w ethyl acetate; and
2% w/w amyl acetate.

15. The composition of claim 11, wherein the composition consists of
6-8% w/w ciclopirox or a salt;
4-5% w/w efinaconazole or a salt;
15-20% w/w ethanol;
10-33% w/w propylene glycol;
10-15% w/w ethyl acetate;
2% w/w amyl acetate; and
30-40% w/w dimethyl sulfoxide.

16. A method of treating onychomycosis in a patient suffering from the disease comprises topically administering a composition comprising ciclopirox and efinaconazole to at least one toe or fingernail of the patient.

17. The method of claim 16, wherein the topically administered composition consists of
4-10% w/w ciclopirox or a salt;
2-8% w/w efinaconazole or a salt;
10-30% w/w ethanol;
0-40% w/w isopropyl alcohol;
10-33% w/w propylene glycol;
5-20% w/w ethyl acetate;
2% w/w amyl acetate; and
0-40% w/w dimethyl sulfoxide.

18. A method of delivering ciclopirox and efinaconazole or salts thereof from the dorsal layer of the nail plate to the nail bed, the method comprises contacting said cell with the combination of ciclopirox and efinaconazole or salts thereof capable of penetrating the nail plate, under conditions sufficient to penetrate said nail plate, and thereby delivering said combination.

19. The method of claim 18, wherein the topically administered combination comprises a composition comprising: (a) a solvent that is volatile and/or that rapidly penetrates a nail following application onto the surface of the nail, (b) a wetting agent, and (c) optionally a non-volatile solvent that is dissolved, suspended, dispersed, or emulsified within the solvent.

20. The method of claim 18, wherein the topically administered combination consists of
4-10% w/w ciclopirox or a salt;
2-8% w/w efinaconazole or a salt;
10-30% w/w ethanol;
0-40% w/w isopropyl alcohol;
10-33% w/w propylene glycol;
5-20% w/w ethyl acetate;
2% w/w amyl acetate; and
0-40% w/w dimethyl sulfoxide.
